(19) United States
(12) Patent Application Publication
(10) Pub. No.: US 2007/0048796 A1
(43) Pub. Date: Mar. 1, 2007

(54) Biosensor

(75) Inventors: Toshiaki Kubo, Asaka-shi (JP); Toshihide Ezoe, Ashigarakami-gun (JP); Hirohiko Tsuzuki, Ashigarakami-gun (JP); Morihito Ikeda, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/508,904

(22) Filed: Aug. 24, 2006

(30) Foreign Application Priority Data
Aug. 26, 2005 (JP) 245442/2005

(51) Int. Cl. G01N 33/53; C12M 1/34

(52) U.S. Cl. 435/7.1; 435/287.2; 977/902; 436/86

(57) ABSTRACT

It is an object of the present invention to provide a biosensor with an improved recovery yield of a test substance interacting with a physiologically active substance. The present invention provides a biosensor having a flow channel formed on a substrate, which is composed of a detection surface for detecting the interaction of a physiologically active substance with a test substance and a non-detection surface that does not detect said interaction, wherein the detection surface and non-detection surface are modified in such a way that the physiologically active substance can be immobilized thereon.

### TECHNICAL INVENTION

The present invention relates to a biosensor, and a method of analyzing interaction among biomolecules using the above biosensor, so as to recover a substance which interacts with a biomolecule. In particular, the present invention relates to a biosensor used for surface plasmon resonance biosensors, and a method of analyzing interaction among biomolecules using the above biosensor, so as to recover a substance which interacting with a biomolecule.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The QCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

As a thin film having a functional group capable of immobilizing a physiologically active substance, there has been reported a measurement chip where a physiologically active substance is immobilized by using a functional group binding to metal, a linker with a chain length of 10 or more atoms, and a compound having a functional group capable of binding to the physiologically active substance (Japanese Patent No. 2815120). Moreover, a measurement chip comprising a metal film and a plasma-polymerized film formed on the metal film has been reported (Japanese Patent Laid-Open (Kokai) No. 9-264843).

A system for combining SPR with other analytical methods has been developed in order to identify a substance interacting with a physiologically active substance on a measurement chip and to obtain the structural information thereof. Surface plasmon resonance mass spectrometry developed by combining mass spectrometry with SPR has been reported as such an effective analytical method (JP Patent Publication (Kohyo) No. 11-512518 A (1999)). This is a method, which comprises analyzing interaction on a measurement chip, directly dropping matrix onto the measurement chip for crystallization, applying laser thereto, and measuring the mass of a molecule interacting with a physiologically active substance on the chip. Otherwise, a substance interacting with a physiologically active substance is recovered from the surface of the measurement chip, and it is then analyzed with a mass spectrometer. However, the use of such methods for the analysis of a test substance has been problematic. There are cases where the amount of a test substance captured on a measurement chip is insufficient for certain mass spectrometers. That is to say, in order to easily detect, measure, and identify a substance interacting with a physiologically active substance, it has been desired that an analytical system that brings on a high yield of a test substance be constructed.

### DISCLOSURE OF INVENTION

It is an object of the present invention to solve the aforementioned problems of the prior art techniques. That is, it is an object of the present invention to provide a biosensor with an improved recovery yield of a test substance interacting with a physiologically active substance.

As a result of intensive studies directed towards achieving the aforementioned object, the present inventors have found that the detection surface and non-detection surface of a flow channel are modified in such a way that a physiologically active substance can be immobilized thereon, thereby providing a biosensor with an improved recovery yield of a substance interacting with the physiologically active substance, thereby completing the present invention.

That is, the present invention provides a biosensor having a flow channel formed on a substrate, which is composed of a detection surface for detecting the interaction of a physiologically active substance with a test substance and a non-detection surface that does not detect the aforementioned interaction, wherein the detection surface and non-detection surface are modified in such a way that the physiologically active substance can be immobilized thereon.

Preferably, the detection surface and non-detection surface of a flow channel are modified with a polymer compound.

Preferably, the polymer compound is a hydrophobic polymer or a hydrophilic polymer.

Preferably, the biosensor of the present invention further has a mechanism for recovering a substance interacting with a physiologically active substance.

Preferably, the substrate is composed of a metal surface or metal film.

Preferably, the metal surface or metal film consists of a free electron metal selected from the group consisting of gold, silver, copper, platinum, and aluminum.

Preferably, the biosensor of the present invention is used in non-electrochemical detection, and is more preferably used in surface plasmon resonance analysis.

In another aspect, the present invention provides a method for producing the biosensor of the present invention, which comprises a step of modifying the detection surface and non-detection surface of a flow channel in such a way that a physiologically active substance can be immobilized thereon.

Preferably, the detection surface and non-detection surface of a flow channel are modified with a polymer compound.

Preferably, the polymer compound is a hydrophobic polymer or a hydrophilic polymer.

In a further aspect, the present invention provides a method for detecting or measuring a substance interacting with a physiologically active substance, which comprises the steps of: allowing the biosensor of the present invention to come into contact with a physiologically active substance, so as to allow the above-described physiologically active substance to bind to the detection surface and non-detection surface of the flow channel of the above-described biosensor via a covalent bond; and allowing a test substance to come into contact with the biosensor, to the detection surface and non-detection surface of the flow channel of which the physiologically active substance has been bound via a covalent bond.

Preferably, the step of allowing a physiologically active substance to bind to a biosensor, and the step of allowing a test substance to come into contact with the biosensor so as to detect or measure a substance interacting with the physiologically active substance, are carried out using different devices.

Preferably, a substance interacting with the physiologically active substance is detected or measured by non-electrochemical detection, and is more preferably detected or measured by surface plasmon resonance analysis.

In a further aspect, the present invention provides a method for analyzing a substance interacting with a physiologically active substance, which comprises identifying and recovering a substance interacting with a physiologically active substance using the biosensor of the present invention, and determining the structure of the recovered substance using a mass spectrometer.

### BRIEF DESCRIPTION OF THE DRAWINGS

FIG. 1 shows an example of the flow channel of the present invention.

### BEST MODE FOR CARRYING OUT THE INVENTION

The embodiments of the present invention will be described below.

The biosensor of the present invention is composed of a substrate and a flow channel formed thereon. The biosensor of the present invention has as broad a meaning as possible, and the term biosensor is used herein to mean a sensor, which converts an interaction between biomolecules into a signal such as an electric signal, so as to measure or detect a target substance. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

The structure of the flow channel used in the present invention is not particularly limited, as long as it is formed on a substrate such that it feeds a liquid. The flow channel in the present invention is composed of a detection surface for detecting the interaction of a physiologically active substance with a test substance and a non-detection surface that does not detect the aforementioned interaction. In addition, the shape of the cross section of the flow channel is not particularly limited, and it may have any given shape such as a square, rectangle, trapezoid, circle, semicircle, or ellipse.

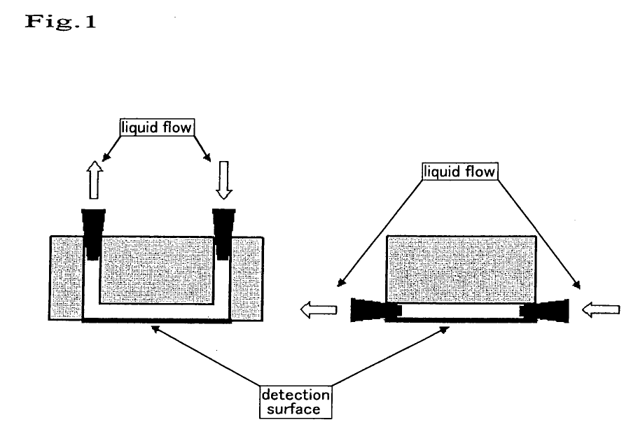

The flow channel in the present invention does not include a syringe or pipette for supplying an agent or protein. However, from the viewpoint of prevention of contamination, it is preferable to use a disposable pipette to supply an agent or protein. An example of the flow channel in the present invention is shown in FIG. 1.

In the left view of FIG. 1, the flow channel is formed with three regions, namely, a region for supplying a liquid, a region including a detection surface, and a region for discharging the liquid. The region for supplying a liquid and the region for discharging the liquid are formed in a direction almost perpendicular to the region including a detection surface. In the case of the left view of FIG. 1, with regard to the region for supplying a liquid and the region for discharging the liquid, all the inner surfaces of the above flow channel become non-detection surfaces. With regard to the region including a detection surface, the bottom surface of the flow channel becomes a detection surface, and the side surface and upper surface thereof become non-detection surfaces.

In the right view of FIG. 1, a flow channel is formed in a straight line. In this case, the bottom surface of the flow channel becomes a detection surface, and the side surface and the upper surface of the flow channel become non-detection surfaces.

A material for the flow channel used in the present invention is not particularly limited. Examples of such a material may include polydimethylcyclohexane, polypropylene, polyethylene, polymethyl methacrylate, and polystyrene.

The term “detection surface” is used in the present specification to mean a surface out of the inner surfaces of a flow channel, on which the interaction of a physiologically active substance with a test substance is detected. In addition, the term “non-detection surface” is used in the present specification to mean a surface out of the inner surfaces of the flow channel, on which the aforementioned interaction is not detected.

Preferably, a mechanism for recovering a substance interacting with a physiologically active substance is further provided in the biosensor of the present invention. As such a mechanism, a pipette or the like can be used.

In the present invention, the aforementioned detection surface and non-detection surface are modified in such a way that the surfaces can immobilize a physiologically active substance thereon. The term “modification” is used herein to preferably mean modification with a polymer compound. As the polymer compound, a hydrophobic polymer or a hydrophilic polymer can be used. Hereafter, the polymer compound which can be used in the present invention will be described.

A hydrophobic polymer used in the present invention is a polymer having no water-absorbing properties. Its solubility in 100 g of water (25° C.) is 10 g or less, more preferably 1 g or less, and most preferably 0.1 g or less.

A hydrophobic monomer which forms a hydrophobic polymer can be selected from vinyl esters, acrylic esters, methacrylic esters, olefins, styrenes, crotonic esters, itaconic diesters, maleic diesters, fumaric diesters, allyl compounds, vinyl ethers, vinyl ketones, or the like. The hydrophobic polymer may be either a homopolymer consisting of one type of monomer, or copolymer consisting of two or more types of monomers.

Examples of a hydrophobic polymer that is preferably used in the present invention may include polystyrene, polyethylene, polypropylene, polyester (such as polyethylene terephthalate), polyvinyl chloride, polymethyl methacrylate, and nylon. A hydrophobic polymer containing styrene is most preferred.

A substrate is coated with a hydrophobic polymer according to common methods. Examples of such a coating method may include spin coating, air knife coating, bar coating, blade coating, slide coating, curtain coating, spray method, evaporation method, cast method, and dip method.

The modification thickness of a hydrophobic polymer is not particularly limited, but it is preferably between 0.1 nm and 500 nm, and particularly preferably between 1 nm and 300 nm.

In the case of the biosensor of the present invention comprising a flow channel modified with a hydrophobic polymer, it has a functional group capable of immobilizing a physiologically active substance on the outermost surfaces of the detection surface and non-detection surface of the flow channel. The expression “the outermost surfaces of the detection surface and non-detection surface of the flow channel” is used herein to mean “the sides farthest from the detection surface and non-detection surface of the flow channel.” More specifically, it means “the sides in a polymer modified on the detection surface and non-detection surface of the flow channel, which are farthest from the detection surface and non-detection surface of the flow channel.”

Specific examples of a functional group for binding a physiologically active substance may include —COOH, —NR¹R² (wherein each of R¹ and R² independently represents a hydrogen atom or a lower alkyl group), —OH, —SH, —CHO, —NR³NR¹R² (wherein each of R¹, R², and R³ independently represents a hydrogen atom or a lower alkyl group), —NCO, —NCS, an epoxy group, and a vinyl group. Herein, the number of carbon atoms contained in a lower alkyl group is not particularly limited, but it is generally approximately C1 to C10, and preferably C1 to C6.

An example of a hydrophilic polymer used in the present invention is a biocompatible porous matrix such as a hydrogel. The thickness of such a biocompatible porous matrix is between several nm and several hundreds of nm, and preferably between 10 and 500 nm. An example of a hydrogel, which can be used in the present invention, is a hydrogel defined in Merrill et al. (1986), Hydrogels in Medicine and Pharmacy, vol. III, edited by Peppas N A, Chapter 1, CRC. Specific examples of a hydrogel, which can be used in the present invention, may include: polysaccharides such as agarose, dextran, carragheenan, alginic acid, starch, cellulose, or a derivative thereof such as a carboxymethyl derivative; and water-swelling organic polymers such as polyvinyl alcohol, polyacrylic acid, polyacrylamide, or polyethylene glycol. A polyethylene glycol derivative and a dextran derivative are particularly preferably used. Most preferably, carboxymethyl dextran is used.

In the present invention, a self-assembled monolayer (SAM) is first formed on a detection surface, and it can be then modified with a hydrophilic polymer. The term “self-assembled monolayer” is used in the present invention to mean an ultra-thin film consisting of tissues with a certain system, which is formed by the mechanism of a film material itself in a state where no detailed control is given from the outside, such as a monomolecular film or an LB film. By such self-assembling, a structure or pattern with a certain system is formed in a nonequilibrium state over a long distance.

For example, the self-assembled monolayer can be formed with a sulfur-containing compound. Formation of a self-assembled monolayer on a gold surface with a sulfur-containing compound is described in Nuzzo R G et al. (1983), J Am Chem Soc, vol. 105, pp. 4481-4483; Porter M D et al. (1987), J Am Chem Soc, vol. 109, pp. 3559-3568; and Troughton E B et al. (1988), Langmuir, vol. 4, pp. 365-385, for example.

In the case of the biosensor of the present invention comprising a flow channel modified with a hydrophilic polymer, it has a functional group capable of immobilizing a physiologically active substance on the outermost surfaces of the detection surface and non-detection surface of the flow channel.

Specific examples of a functional group for binding a physiologically active substance may include —COOH, —NR¹R² (wherein each of R¹ and R² independently represents a hydrogen atom or a lower alkyl group), —OH, —SH, —CHO, —NR³NR¹R² (wherein each of R¹, R², and R³ independently represents a hydrogen atom or a lower alkyl group), —NCO, —NCS, an epoxy group, and a vinyl group. Herein, the number of carbon atoms contained in a lower alkyl group is not particularly limited, but it is generally approximately C1 to C10, and preferably C1 to C6. Particularly preferred are —COOH, —NH₂, —CHO, —NHNH₂, an epoxy group, and a vinyl group.

In the present invention, as a method of modifying the non-detection surface of a flow channel, there is used a method of coating the non-detection surface of a flow channel with gold via evaporation and then forming a hydrophobic polymer film by the dip and adsorption method described in Japanese Patent Application Laid-Open No. 2005-189222, 2004-271514, and the like, or a method of forming a self-assembled monolayer as in the case of a detection surface and then binding a hydrophilic polymer thereto. Moreover, it is also possible that using a silane coupling agent, a hydroxyl group be allowed to generate only on the non-detection surface, and thereafter, the same treatment as that for the detection surface be performed thereon.

In the dip and adsorption method, coating is carried out by a method comprising allowing a substrate to come into contact with a hydrophobic polymer solution, and then allowing it to come into contact with a solution that does not contain the aforementioned hydrophobic polymer. A solvent in the hydrophobic polymer solution is preferably identical to a solvent in the solution that does not contain a hydrophobic polymer.

In the dip method, a layer of a hydrophobic polymer having an uniform coating thickness can be obtained on a surface of a substrate regardless of inequalities, curvature and shape of the substrate by suitably selecting a coating solvent for hydrophobic polymer.

The type of coating solvent used in the dip method is not particularly limited, and any solvent can be used so long as it can dissolve a part of a hydrophobic polymer. Examples thereof include formamide solvents such as N,N-dimethylformamide, nitrile solvents such as acetonitrile, alcohol solvents such as phenoxyethanol, ketone solvents such as 2-butanone, and benzene solvents such as toluene, but are not limited thereto.

In the solution of a hydrophobic polymer which is contacted with a substrate, the hydrophobic polymer may be dissolved completely, or alternatively, the solution may be a suspension which contains undissolved component of the hydrophobic polymer. The temperature of the solution is not particularly limited, so long as the state of the solution allows a part of the hydrophobic polymer to be dissolved. The temperature is preferably −20° C. to 100° C. The temperature of the solution may be changed during the period when the substrate is contacted with a solution of a hydrophobic polymer. The concentration of the hydrophobic polymer in the solution is not particularly limited, and is preferably 0.01% to 30%, and more preferably 0.1% to 10%.

The period for contacting the solid substrate with a solution of a hydrophobic polymer is not particularly limited, and is preferably 1 second to 24 hours, and more preferably 3 seconds to 1 hour.

As the liquid which does not contain the hydrophobic polymer, it is preferred that the difference between the SP value (unit: (J/cm³)^{1/2}) of the solvent itself and the SP value of the hydrophobic polymer is 1 to 20, and more preferably 3 to 15. The SP value is represented by a square root of intermolecular cohesive energy density, and is referred to as solubility parameter. In the present invention, the SP value δ was calculated by the following formula. As the cohesive energy (Ecoh) of each functional group and the mol volume (V), those defined by Fedors were used (R. F. Fedors, Polym. Eng. Sci., 14(2), P147, P472(1974)).
Δ=(ΣEcoh/ΣV)^{1/2}

Examples of the SP values of the hydrophobic polymers and the solvents are shown below;
Solvent: 2-phenoxyethanol: 25.3 against polymethylmethacrylate-polystyrene copolymer (1:1): 21.0
Solvent: acetonitrile: 22.9 against polymethylmethacrylate: 20.3
Solvent: toluene: 18.7 against polystyrene: 21.6

The period for contacting a substrate with a liquid which does not contain the hydrophobic polymer is not particularly limited, and is preferably 1 second to 24 hours, and more preferably 3 seconds to 1 hour. The temperature of the liquid is not particularly limited, so long as the solvent is in a liquid state, and is preferably −20° C. to 100° C. The temperature of the liquid may be changed during the period when the substrate is contacted with the solvent. When a less volatile solvent is used, the less volatile solvent may be substituted with a volatile solvent which can be dissolved in each other after the substrate is contacted with the less volatile solvent, for the purpose of removing the less volatile solvent.

The non-detection surface of the flow channel of the present invention may be subjected to either the same modification as that for the detection surface, or modification different therefrom. However, it is preferable that the non-detection surface be subjected to the same modification as that for the detection surface.

The biosensor of the present invention is preferably obtained by coating a metal surface or metal film with a hydrophobic polymer or a hydrophilic polymer. A metal constituting the metal surface or metal film is not particularly limited, as long as surface plasmon resonance is generated when the metal is used for a surface plasmon resonance biosensor. Examples of a preferred metal may include free-electron metals such as gold, silver, copper, aluminum or platinum. Of these, gold is particularly preferable. These metals can be used singly or in combination. Moreover, considering adherability to the above substrate, an interstitial layer consisting of chrome or the like may be provided between the substrate and a metal layer.

The film thickness of a metal film is not limited. When the metal film is used for a surface plasmon resonance biosensor, the thickness is preferably between 0.1 nm and 500 nm, more preferably between 0.5 nm and 500 nm, and particularly preferably between 1 nm and 200 nm. If the thickness exceeds 500 nm, the surface plasmon phenomenon of a medium cannot be sufficiently detected. Moreover, when an interstitial layer consisting of chrome or the like is provided, the thickness of the interstitial layer is preferably between 0.1 nm and 10 nm.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and have excellent workability are preferably used.

A physiologically active substance immobilized on the detection surface and non-detection surface of the flow channel of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against O antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

When a physiologically active substance is a protein such as an antibody or enzyme or nucleic acid, an amino group, thiol group or the like of the physiologically active substance is covalently bound to a functional group located on a metal surface, so that the physiologically active substance can be immobilized on the metal surface.

A biosensor to which a physiologically active substance is immobilized as described above can be used to detect and/or measure a substance which interacts with the physiologically active substance.

Further, a substance interacting with the physiologically active substance which was bound to the detection surface and non-detection surface of the flow channel, can be recovered.

Thus, the present invention provides a method of detecting and/or measuring and/or recovering a substance interacting with a physiologically active substance, which comprises a step of allowing a test substance to come into contact with the biosensor of the present invention to which the physiologically active substance has been bound.

As such a test substance, for example, a sample containing the above substance interacting with the physiologically active substance can be used.

In the present invention, it is preferable to detect and/or measure an interaction between a physiologically active substance immobilized on the surface used for a biosensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance which is characterized in that it comprises a metal film placed on a transparent substrate.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

When the biosensor of the present invention is used in surface plasmon resonance analysis, it can be adopted as a part of various types of surface plasmon measurement devices described in paragraphs [0041] to [0054] of JP Patent Publication (Kokai) No. 2004-271514 A.

Furthermore, it is also possible that a substance interacting with a physiologically active substance be identified and recovered using the biosensor of the present invention, and the structure of the recovered substance be then determined using a mass spectrometer. As a mass spectrometer, MALDI (Matrix Assisted Laser Desorption/Ionization) or the like can be used. Further, it is also possible that the recovered substance be digested with protease, that the mass spectrometric spectrum of a peptide be obtained, and that the obtained spectrum be then certified by comparing with the mass spectrometric spectrum of the previously known protein or the mass spectrometric spectrum predicted from genomic information, thereby identifying a protein interacting with the physiologically active substance.

The present invention will be further described in the following examples. However, these examples are not intended to limit the scope of the present invention.

### EXAMPLES

### Example 1

### Production of Biosensor of the Present Invention Coated with Hydrophilic Surface

The sensor chip and flow channel of the present invention were produced by the following method.

A polycycloolefin prism and a polypropylene flow channel (the left view of FIG. 1) were coated with gold via evaporation, and they were then coated with a hydrophilic compound.

(1) Gold Evaporation

The polycycloolefin prism and the polypropylene flow channel were attached to the substrate holder of a sputter device. After decompression (base pressure: 1×10⁻³ Pa or less), Ar gas (1 Pa) was introduced therein. Thereafter, while rotating the substrate holder (20 rpm), RF power (0.5 kW) was applied to the substrate holder for approximately 9 minutes, so as to subject FET to a plasma treatment (which is also referred to as substrate etching or reverse sputtering). Subsequently, introduction of Ar gas was terminated, followed by decompression. Thereafter, Ar gas was introduced again (0.5 Pa), and while rotating the substrate holder (10 to 40 rpm), DC power (0.2 kW) was applied to a Cr target having a size of 8 inch for approximately 30 seconds, so as to form a thin Cr film having a thickness of 2 nm. Subsequently, introduction of Ar gas was terminated, followed by decompression again. Thereafter, Ar gas was introduced again (0.5 Pa), and while rotating the substrate holder (20 rpm), DC power (1 kW) was applied to an Au target having a size of 8 inch for approximately 50 seconds, so as to form a thin Au film having a thickness of approximately 50 nm. The particle size of Au was approximately 20 nm.

(2) Production of Hydrogel Layer

Preparation of Solutions

SAM Solution:

A SAM solution was produced by fully mixing 0.0102 g of 11-hydroxy-1-undecanethiol (manufactured by Dojindo Laboratories), 2 ml of ultrapure water, and 8 ml of ethanol.

Epichlorohydrin Solution:

An epichlorohydrin solution was produced by fully mixing 500 μl of epichlorohydrin (manufactured by Wako Pure Chemical Industries, Ltd.), 4.5 ml of diethylene glycol dimethyl ether, 3 ml of ultrapure water, and 2 ml of 1 mol/L NaOH.

Dextran Solution:

A dextran solution was produced by fully mixing 3 g of dextran 500 (manufactured by Amersham), 9 ml of ultrapure water, and 1 ml of 1 mol/L NaOH.

Bromoacetic Acid Solution:

A bromoacetic acid solution was produced by fully mixing 1.2 g of bromoacetic acid, 5.4 ml of ultrapure water, and 3.2 ml of 5 mol/l NaOH.

Operations

The SAM solution was allowed to come into contact with the polycycloolefin prism surface and polypropylene flow channel surface, which had been coated with gold via evaporation, so that they were reacted at 40° C. for 30 minutes. Thereafter, the reaction was further carried out at room temperature for 16 hours. Thereafter, the surfaces were washed, and the epichlorohydrin solution was then allowed to come into contact with the surfaces, so that they were reacted at room temperature for 8 hours. Thereafter, the surfaces were washed, and the dextran solution was then allowed to come into contact with the surfaces, so that they were reacted at room temperature for 16 hours. Thereafter, the surfaces were washed. The bromoacetic acid solution was further allowed to come into contact with the surfaces at room temperature for 24 hours. Thereafter, the surfaces were washed, and the bromoacetic acid solution was again allowed to come into contact with the surfaces for 24 hours, followed by washing.

### Example 2

### Evaluation of Performance of Biosensor of the Present Invention Coated with Hydrophilic Surface

Measurement of Binding and Recovery of Protein

A biosensor produced by the combination of a measurement surface with the flow channel of the present invention and a biosensor produced by the combination of a measurement surface with an untreated flow channel were used to conduct binding measurement and a recovery experiment.

Operations

(1) Preparation of Ligand Solution:

0.5 mg of an anti-BSA (bovine serum albumin) antibody (manufactured by Rockland) was dissolved in 1 ml of an acetate buffer (pH 5.5).

(2) Preparation of Activator Solution:

The following solutions were mixed with each other at a volume ratio of 1:1, immediately before use: 0.1 M NHS solution and 0.4 M EDC solution.

(3) Blocking Solution: 1 M Ethanolamine Solution (pH 8.5)

(4) Analyte Solution:

1 mg of BSA (manufactured by Sigma) was dissolved in 1 ml of HBS-EP buffer. The HBS-EP buffer consisted of 0.01 mol/l HEPES (N-2-hydroxyethylpiperazine-N′-2-ethanesulfonic acid) (pH 7.4), 0.15 mol/l NaCl, 0.003 mol/l EDTA, and 0.005% by weight of Surfactant P20.

A chip was set in a device, and the flow channel thereof was filled with the HBS-EP buffer. The measurement was initiated in such a state, and the signal value obtained 30 seconds after initiation of the measurement was defined as 0. While the measurement was continued, 100 μl of the activator solution was poured into the flow channel for 1 second, and it was then left for 15 minutes. Subsequently, 100 μl of the HBS-EP buffer was poured into the flow channel for 1 second, and 100 μl of the ligand solution was then poured therein for 1 second. It was left for 15 minutes. Thereafter, 100 μl of the HBS-EP buffer was poured into the flow channel for 1 second, and 100 μl of the blocking solution was then poured therein for 1 second. It was left for 15 minutes. Thereafter, the operation to pour 100 μl of the HBS-EP buffer into the flow channel for 1 second and then to pour 100 μl of a 10 mM NaOH solution therein for 1 second was repeated twice, followed by substitution with HBS-EP. The resultant was then left for 30 seconds. The signal value obtained at that time was defined as the amount of a ligand immobilized.

The above chip was still set in the above device, and the analyte was measured. The flow channel was filled with the HBS-EP buffer, and the measurement was initiated in such a state. The signal value obtained 60 seconds after initiation of the measurement was defined as 0. While the measurement was continued, 100 μl of the analyte solution was poured into the flow channel for 1 second, and it was then left for 3 minutes. The signal value obtained 3 minutes later was measured. Further, 100 μl of the HBS-EP buffer was poured into the flow channel for 1 second, and thereafter, the flow channel was filled with 50 mM NaOH aqueous solution, followed by leaving it for 180 seconds. Thereafter, the NaOH aqueous solution filled in the flow channel was recovered. The recovered solution was subjected to decompression concentration, so as to exsiccate a solid. Thereafter, it was diluted with 10 μl of the HBS-EP buffer. Absorption at 280 nm was measured with ND-1000 (NanoDrop), and the obtained value was defined as the amount of a protein recovered.

(2) Results

Table 1 shows the results regarding the measurement of the amount of a protein bound and the amount of a protein recovered.

TABLE 1

| | | | Amount | |
|---|---|---|---|---|
| | | Amount | of BSA | |
| Flow | Measurement | of BSA | recovered | |
| channel | surface | bound | Abs (280 nm) | Remarks |
| With surface | With surface | 5000 RU | 0.95 | The present |
| modification | modification | | | invention |
| Without | With surface | 5000 RU | 0.12 | Comparative |
| surface | modification | | | example |
| modification | | | | |

From the results shown in Table 1, it was found that the biosensor of the present invention enables a significant increase in the recovered amount of a sample detected. That is to say, a biosensor with excellent recovery ability could be provided.

### Example 3

### Production of Biosensor of the Present Invention Coated with a Hydrophobic Surface

The sensor chip and flow channel of the present invention were produced by the following method.

A polycycloolefin prism and a polypropylene flow channel were coated with gold via evaporation, and were then coated with a hydrophobic compound.

(1) Gold Evaporation

The polycycloolefin prism and the polypropylene flow channel were attached to the substrate holder of a sputter device. After decompression (base pressure: 1×10⁻³ Pa or less), Ar gas (1 Pa) was introduced therein. Thereafter, while rotating the substrate holder (20 rpm), RF power (0.5 kW) was applied to the substrate holder for approximately 9 minutes, so as to subject FET to a plasma treatment (which is also referred to as substrate etching or reverse sputtering). Subsequently, introduction of Ar gas was terminated, followed by decompression. Thereafter, Ar gas was introduced again (0.5 Pa), and while rotating the substrate holder (10 to 40 rpm), DC power (0.2 kW) was applied to a Cr target having a size of 8 inch for approximately 30 seconds, so as to form a thin Cr film having a thickness of 2 nm. Subsequently, introduction of Ar gas was terminated, followed by decompression again. Thereafter, Ar gas was introduced again (0.5 Pa), and while rotating the substrate holder (20 rpm), DC power (1 kW) was applied to an Au target having a size of 8 inch for approximately 50 seconds, so as to form a thin Au film having a thickness of approximately 50 nm. The particle size of Au was approximately 20 nm.

(2) Production of Polymer Layer

A polymethyl methacrylate-polystyrene copolymer (PMMA/PSt) (molar ratio 50:50; mean molecular weight: 20000) was applied onto the surface coated with gold via evaporation by the method described in Japanese Patent Application No. 2003-405704, resulting in a film thickness of 20 nm. That is to say, a gold block was treated with a Model-208 UV-ozone cleaning system (TECHNOVISION INC.) for 30 minutes, and thereafter, 1% PMMA/PSt was added dropwise to the surface coated with gold via evaporation, followed by leaving it at rest for 15 minutes. Thereafter, the surface was immersed in 50 ml of N,N-dimethylformamide for 1 minute 5 times, so as to substitute 1% PMMA/PSt on the surface coated with gold via evaporation with N,N-dimethylformamide. After such substitution, N,N-dimethylformamide on the block surface was eliminated by nitrogen blowing, and it was then dried for 16 hours under decompression. The thickness of the film was measured by the ellipsometry method (In-Situ Ellipsometer MAUS-101, manufactured by Five Lab). As a result, the thickness of the PMMA/PSt film was found to be 20 nm. Thereafter, hydrolysis was carried out under the conditions described in Japanese Patent Application No. 2003-405704 (that is, the surface was immersed in an NaOH aqueous solution (1 N) at 40° C. for 16 hours, and it was then washed with water 3 times, followed by elimination of the water by nitrogen blowing), so as to generate carboxylic acid. The surface coated with the generated carboxylic acid was immersed in a mixed solution of 1-ethyl-2,3-dimethylaminopropylcarbodiimide (400 nM) and N-hydroxysuccinimide (100 mM) for 60 minutes. The surface was then immersed in a 5-aminovaleric acid (1 mol/l; adjusted to pH 8.5) solution for 16 hours, followed by washing.

### Example 4

### Evaluation of Performance of Biosensor of the Present Invention Coated with Hydrophilic Surface

Measurement of Binding and Recovery of Protein

A biosensor produced by the combination of a measurement surface with the flow channel of the present invention and a biosensor produced by the combination of a measurement surface with an untreated flow channel were used to conduct binding measurement and a recovery experiment.

Operations

(1) Preparation of Ligand Solution:

0.5 mg of an anti-BSA (bovine serum albumin) antibody (manufactured by Rockland) was dissolved in 1 ml of an acetate buffer (pH 5.5).

(2) Preparation of Activator Solution:

The following solutions were mixed with each other at a volume ratio of 1:1, immediately before use: 0.1 M NHS solution and 0.4 M EDC solution.

(3) Blocking Solution: 1 M Ethanolamine Solution (pH 8.5)

(4) Analyte Solution:

1 mg of BSA (manufactured by Sigma) was dissolved in 1 ml of the HBS-EP buffer.

A chip was set in a device, and the flow channel thereof was filled with an HBS-EP buffer. The measurement was initiated in such a state, and the signal value obtained 30 seconds after initiation of the measurement was defined as 0. While the measurement was continued, 100 μl of the activator solution was poured into the flow channel for 1 second, and it was then left for 15 minutes. Subsequently, 100 μl of the HBS-EP buffer was poured into the flow channel for 1 second, and 100 μl of the ligand solution was then poured therein for 1 second. It was left for 15 minutes. Thereafter, 100 μl of the HBS-EP buffer was poured into the flow channel for 1 second, and 100 μl of the blocking solution was then poured therein for 1 second. It was left for 15 minutes. Thereafter, the operation to pour 100 μl of the HBS-EP buffer into the flow channel for 1 second and then to pour 100 μl of a 10 mM NaOH solution therein for 1 second was repeated twice, followed by substitution with HBS-EP. The resultant was then left for 30 seconds. The signal value obtained at that time was defined as the amount of a ligand immobilized.

The above chip was still set in the above device, and the analyte was measured. The flow channel was filled with the HBS-EP buffer, and the measurement was initiated in such a state. The signal value obtained 60 seconds after initiation of the measurement was defined as 0. While the measurement was continued, 100 μl of the analyte solution was poured into the flow channel for 1 second, and it was then left for 3 minutes. The signal value obtained 3 minutes later was measured. Further, 100 μl of the HBS-EP buffer was poured into the flow channel for 1 second, and thereafter, the flow channel was filled with 50 mM NaOH aqueous solution, followed by leaving it for 180 seconds. Thereafter, the NaOH aqueous solution filled in the flow channel was recovered. The recovered solution was subjected to decompression concentration, so as to exsiccate a solid. Thereafter, it was diluted with 10 μl of the HBS-EP buffer. Absorption at 280 nm was measured with ND-1000 (NanoDrop), and the obtained value was defined as the amount of a protein recovered.

(2) Results

Table 2 shows the results regarding the measurement of the amount of a protein bound and the amount of a protein recovered.

TABLE 2

| | | | Amount | |
|---|---|---|---|---|
| | | Amount | of BSA | |
| Flow | Measurement | of BSA | recovered | |
| channel | surface | bound | Abs (280 nm) | Remarks |
| With surface | With surface | 1500 RU | 0.24 | The present |
| modification | modification | | | invention |
| Without | With surface | 1500 RU | 0.03 | Comparative |
| surface | modification | | | example |
| modification | | | | |

From the results shown in Table 2, it was found that the biosensor of the present invention enables a significant increase in the recovered amount of a sample detected. That is to say, a biosensor with excellent recovery ability could be provided.

### EFFECTS OF THE INVENTION

According to the present invention, it became possible to provide a biosensor having an improved recovery yield of a test substance interacting with a physiologically active substance.

1. A biosensor having a flow channel formed on a substrate, which is composed of a detection surface for detecting the interaction of a physiologically active substance with a test substance and a non-detection surface that does not detect said interaction, wherein the detection surface and non-detection surface are modified in such a way that the physiologically active substance can be immobilized thereon.

2. The biosensor according to claim 1 wherein the detection surface and non-detection surface of a flow channel are modified with a polymer compound.

3. The biosensor according to claim 2 wherein the polymer compound is a hydrophobic polymer.

4. The biosensor according to claim 2 wherein the polymer compound is a hydrophilic polymer.

5. The biosensor according to claim 1 which further has a mechanism for recovering a substance interacting with a physiologically active substance.

6. The biosensor according to claim 1 wherein the substrate is composed of a metal surface or metal film.

7. The biosensor according to claim 1 wherein the metal surface or metal film consists of a free electron metal selected from the group consisting of gold, silver, copper, platinum, and aluminum.

8. The biosensor according to claim 1 which is used in non-electrochemical detection.

9. The biosensor according to claim 1 which is used in surface plasmon resonance analysis.

10. A method for producing the biosensor of claim 1, which comprises a step of modifying the detection surface and non-detection surface of a flow channel in such a way that a physiologically active substance can be immobilized thereon.

11. The method according to claim 10 wherein the detection surface and non-detection surface of a flow channel are modified with a polymer compound.

12. The method according to claim 11 wherein the polymer compound is a hydrophobic polymer.

13. The method according to claim 11 wherein the polymer compound is a hydrophilic polymer.

14. A method for detecting or measuring a substance interacting with a physiologically active substance, which comprises the steps of: allowing the biosensor of claim 1 to come into contact with a physiologically active substance, so as to allow the said physiologically active substance to bind to the detection surface and non-detection surface of the flow channel of said biosensor via a covalent bond; and allowing a test substance to come into contact with the biosensor, to the detection surface and non-detection surface of the flow channel of which the physiologically active substance has been bound via a covalent bond.

15. The method according to claim 14 wherein the step of allowing a physiologically active substance to bind to a biosensor, and the step of allowing a test substance to come into contact with the biosensor so as to detect or measure a substance interacting with the physiologically active substance, are carried out using different devices.

16. The method according to claim 14 wherein a substance interacting with the physiologically active substance is detected or measured by non-electrochemical detection.

17. The method according to claim 14 wherein a substance interacting with the physiologically active substance is detected or measured by surface plasmon resonance analysis.

18. A method for analyzing a substance interacting with a physiologically active substance, which comprises identifying and recovering a substance interacting with a physiologically active substance using the biosensor of claim 1, and determining the structure of the recovered substance using a mass spectrometer.
